(19) United States
(12) Patent Application Publication
(10) Pub. No.: US 2017/0112614 A1
(43) Pub. Date: Apr. 27, 2017

(54) SELF-SUSTAINING ARTIFICIAL COCHLEA

(71) Applicants: Robert A. Goldenberg, Dayton, OH (US); Yan Zhuang, Ceterville, OH (US); Jared Allen Evans, Beavercreek, OH (US)

(72) Inventors: Robert A. Goldenberg, Dayton, OH (US); Yan Zhuang, Ceterville, OH (US); Jared Allen Evans, Beavercreek, OH (US)

(73) Assignee: Wright State University, Dayton, OH (US)

(21) Appl. No.: 15/331,391

(22) Filed: Oct. 21, 2016

Related U.S. Application Data
(60) Provisional application No. 62/245,360, filed on Oct. 23, 2015.

(51) Int. Cl. A61F 2/18; H04R 17/02; H04R 25/00; H04R 17/00

(52) U.S. Cl. CPC A61F 2/18; H04R 17/005; H04R 17/025; H04R 25/606; H04R 25/658; A61F 2002/183; A61F 2240/001

(57) ABSTRACT

Piezoelectric transducer arrays enhanced with flexoelectric effects and a method thereof is disclosed. The array can include an ultrathin piezoelectric ring that includes embedded rings. Adjacent ring transducers can be completely separated from each other on a supporting substrate. The supporting substrate can have cavities to support the transducer array. Additionally, such an array can include electrodes attached to the rings.

### CROSS-REFERENCE TO RELATED APPLICATIONS

This application claims the benefit of U.S. Provisional Patent Application Ser. No. 62/245,360 entitled “SELF-SUSTAINING ARTIFICIAL COCHLEA” filed on Oct. 23, 2015. The entirety of the above-noted application is incorporated herein by reference.

### ORIGIN

The innovation disclosed herein relates to an artificial cochlea and more specifically, to an artificial cochlea that utilizes piezoelectric materials with high flexoelectric properties.

### BACKGROUND

According to the National Center for Health Statistics (HCHS) of the U.S. Department of Health and Human Services, an estimated 20,295,000 people, or 8.6% of the U.S. population 3 years or older, are reported to have hearing problems. Hearing impairment can result from a number of conditions, including hereditary or birth problems, ear infections, head trauma, and otosclerosis, or from age related hearing loss, which is the most common. Excessive or prolonged noise exposure in industry, military, or recreational environments can produce onset of hearing loss symptoms as well.

In light of these auditory statistics, understanding the functionality of the ear is essential to remedying the ailment. The human ear consists of three sections, the outer ear, the middle ear, and the inner ear. The outer ear functions to collect sound waves and funnel them to the middle ear mechanisms. Sound waves travelling through the air are captured by the auricle and intensified by the ear canal, inciting vibrations of the ear drum. These vibrations excite the three bones of the middle ear, the ossicles, which serve to mechanically amplify the sound. The mechanical oscillations transfer the waves to the inner ear.

The inner ear houses the components that convert sound waves to electrical signals and stimulate the hearing centers of the brain. After amplification by the ossicles, compression waves are generated in the extracellular perilymph fluid in the scala tympani and scala vestibuli of the spiral-shaped cochlea. The fluid displacement stimulates a structure called the basilar membrane, housed in the approximately 30 mm length of the spiral structure. The basilar membrane vibrates selectively along its length based on the frequency of the incoming signal; this selectivity is due to its varying stiffness and size. The basilar membrane responds to higher frequencies near the base and lower frequencies near the apex.

As pressure displaces the basilar membrane, the movement stimulates receptors called hair cells, which convert the vibrations to electrical signals. The movement of the inner hair creates a potential difference, between 5-10 millivolts from the resting state, while outer hair cells act to control the total output. The resulting electrical signal stimulates nerve bundles connected to the brain, leading to what is perceived as hearing.

Two classifications can be used to describe hearing loss, regarding which section of the ear is impaired: 1) conduction deafness and 2) sensorineural deafness. Conduction deafness refers to problems originating in the outer and middle ear, including blockage of the ear canal, damage to the tympanic membrane, ear infections, and decreased movement of the ossicles. Sensorineural deafness results from impairment of the hair cells and surrounding structures and the auditory nerve.

Conduction deafness can be treated with hearing aids, which amplify sounds entering the outer ear, or surgical reconstruction of the impaired structures. Sensorineural deafness may be remedied with hearing aids for mild cases or cochlear implants for severe loss. Cochlear implants directly stimulate the nerves of the inner ear using both external and implanted devices. For significant nerve deafness and damage to the inner ear, where hearing aids are not effective to attain useful hearing, cochlear implants can provide a wider range of benefits.

A conventional cochlear implant is a battery-powered, external device that is worn over the ear and attached to the skull. It generally consists of a microphone to receive sound, a signal processor to process sound into electrical signals, external and internal magnetically implanted coils, and electrodes that are inserted into the cochlea to stimulate the cochlear nerve. It has been in clinical use for nearly 30 years. There are some aesthetic and functional concerns about the external portion of the device, and the sound quality of speech and music is not entirely satisfactory. The system also requires that the battery be replaced or recharged.

Conventional cochlear implants from several manufacturers each utilize a similar procedure for digitalizing sound and applying it to the nerves of the inner ear. In contrast, each have different benefits towards size, reliability, electrode quantity, and additional features. The process of implanting the electrode array usually destroys any remnant hearing and therefore is typically only used for extreme cases of hearing.

### SUMMARY

The following presents a simplified summary in order to provide a basic understanding of some aspects of the innovation. This summary is not an extensive overview of the innovation. It is not intended to identify key/critical elements or to delineate the scope of the innovation. Its sole purpose is to present some concepts of the innovation in a simplified form as a prelude to the more detailed description that is presented later.

In one aspect of the innovation, disclosed herein is an innovative self-sustaining artificial cochlea that overcomes the conventional disadvantages mentioned above. The artificial cochlea utilizes piezoelectrics with high flexoelectric properties, which can be self-sustainable and can function naturally with the mechanisms of the human ear. Piezoelectric films emulate the relationship between the basilar membrane and inner hair cell structures of the human cochlear epithelium, inducing a potential difference in response to sound pressure.

In another aspect of the innovation, a hearing device is disclosed that includes a plurality of piezoelectric rings, and a polymeric substrate, wherein the plurality of piezoelectric rings are attached to the polymeric substrate.

In another aspect of the innovation, an artificial cochlea is disclosed and includes a piezoelectric transducer array that includes a plurality of piezoelectric rings, and a polymeric substrate, wherein the plurality of piezoelectric rings are attached to the polymeric substrate.

In still yet another aspect of the innovation, a method is disclosed that includes providing a polymer resin, molding the resin into a predetermined thickness, introducing an electric field to the resin, forming a piezoelectric film, covering the piezoelectric film with a metal layer, and forming the piezoelectric film into a plurality of piezoelectric rings using the metal layer as a first mask.

In another aspect, the subject innovation can comprise a method. Such a method can include the acts of fabricating a piezoelectric device and etching a plurality of connected rings in the piezoelectric device. The method also defines arranging the plurality of rings onto a substrate. Additionally, the method can include attaching a plurality of electrodes to the plurality of rings.

To accomplish the foregoing and related ends, certain illustrative aspects of the innovation are described herein in connection with the following description and the annexed drawings. These aspects are indicative, however, of but a few of the various ways in which the principles of the innovation can be employed and the subject innovation is intended to include all such aspects and their equivalents. Other advantages and novel features of the innovation will become apparent from the following detailed description of the innovation when considered in conjunction with the drawings.

### BRIEF DESCRIPTION OF THE DRAWINGS

The accompanying drawings, which are incorporated in and constitute a part of the specification, illustrate various systems, methods, and other embodiments of the disclosure. Illustrated element boundaries (e.g., boxes, groups of boxes, or other shapes) in the figures represent one example of the boundaries. In some examples one element may be designed as multiple elements or multiple elements may be designed as one element. In some examples, an element shown as an internal component of another element may be implemented as an external component and vice versa.

FIG. 1A-1J are images of a piezoelectric force microscopic measurement of a thin PVDF film with thickness of 20 μm under DC volt bias of 8, 9, 10, 11, 12, −8, −9, −10, −11, and −12 volts respectively where each film is under a force of 500 nN, 1000 nN, and 1500 nN from top to bottom respectively in accordance with an aspect of the innovation.

FIG. 2 is an illustration of an innovative piezoelectric ring array inserted in a human cochlea in accordance with an aspect of the innovation.

FIG. 3 is a perspective view of a portion of the innovative piezoelectric ring array mounted on a substrate in accordance with an aspect of the innovation.

FIG. 4 is a plan view of a piezoelectric ring in accordance with an aspect of the innovation.

FIG. 5 is a flow chart illustrating a method of fabricating a piezoelectric transducer ring array in accordance with an aspect of the innovation.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding of the subject innovation. It may be evident, however, that the innovation can be practiced without these specific details. In other instances, well-known structures and devices are shown in block diagram form in order to facilitate describing the innovation.

While specific characteristics are described herein (e.g., thickness, orientation, configuration, etc.), it is to be understood that the features, functions and benefits of the innovation can employ characteristics that vary from those described herein. These alternatives are to be included within the scope of the innovation and claims appended hereto.

While, for purposes of simplicity of explanation, the one or more methodologies shown herein, e.g., in the form of a flow chart, are shown and described as a series of acts, it is to be understood and appreciated that the subject innovation is not limited by the order of acts, as some acts may, in accordance with the innovation, occur in a different order and/or concurrently with other acts from that shown and described herein. For example, those skilled in the art will understand and appreciate that a methodology could alternatively be represented as a series of interrelated states or events, such as in a state diagram. Moreover, not all illustrated acts may be required to implement a methodology in accordance with the innovation.

Hearing loss is a prevalent issue, affecting all ages in varied occupations. Cochlear implants and hearing aids are a couple of solutions to sensorineural hearing complications. Conventional cochlear implants and hearing aids, however, have limitations in power consumption and also include bulky external equipment that externally attaches to the user's ear and also to the user's head. Disclosed herein is a self-sustaining artificial cochlea utilizing a piezoelectric transducer array that overcomes the above mentioned disadvantages.

Normal hearing occurs when sound enters the external auditory canal and causes a vibration of the tympanic membrane. Sound is then amplified through the middle ear transformer mechanism which in turn creates a traveling wave in the inner ear fluid of the cochlea. This fluid wave stimulates the hair cells of the Organ of Corti. The mechanical excitation creates an electrical charge in each hair cell, which stimulates specific neurons of the auditory nerve; neural stimulation ascends the higher levels of the auditory pathway to the cortex of the brain, where sound is transformed into hearing. Except for individuals who have some form of cognitive impairment, the only defect of the auditory pathway for those with age related or noise induced hearing loss is in the hair cells in the Organ of Corti.

This defect is replaced with an innovative artificial cochlea that utilizes piezoelectric materials. By utilizing all of the otherwise normally functioning components of the auditory pathway, reception of sounds can be much closer to normal hearing than with the innovative cochlear implant without external power. Thus, disclosed herein is an innovative artificial cochlea that uses piezoelectric materials to create transducer ring arrays and methods of making the same, wherein the piezoelectric transducer ring arrays have characteristics of sufficient output to stimulate the inner hair cells and selective response to human speech frequencies based on location. The number of frequency channels in such a piezoelectric transducer array can be based on the quantity of transducer rings mounted on a substrate.

A piezoelectric is a natural transducer, converting stress/strain to electrical signals. Dielectric materials also contain flexoelectric properties, where a strain gradient produces electrical signals, which is amplified as device size is decreased. Therefore, the subject innovation can be manufactured from ultrathin film and utilize a ring structure to improve the non-uniform strain and thus, flexoelectric effects. The piezoelectric transducer rings can be driven by the force of the basilar membrane, giving each transducer equal output along the cochlea. Similar to the frequency selectivity of the basilar membrane due to its stiffness gradient, the piezoelectric film will stimulate select nerves by fabrication of the plurality of segments. If placed in close proximity to the auditory nerves, an output of 1V should be sufficient to stimulate hearing, foregoing the damaged inner ear. Electrodes can be deposited on the ring structures to accumulate charge to stimulate the nerves. The size of the electrodes can vary to ensure the charge density is less than 32 uC/cm² to ensure safety of the relevant cells.

A base of the embodiment can be a piezoelectric sheet, having a thickness of a few hundred nanometers to one micron. Though ceramic piezoelectrics provide high outputs, highly noticeable piezoelectric effect in polymers, such as polyvinylidene difluoride (PVDF), have also been discovered. Polymeric piezoelectrics can provide advantages in this application over ceramics due to their favorable acoustic impedance and flexibility. These polymers could be PVDF, PVDF-TrFE, or any other suitable material with high output and bio-compatibility. In this example embodiment, three major alterations can be made to the original piezoelectric film: 1) etches to define the plurality of ring structures, 2) deposition of top and bottom-side metals, and 3) attachment of ring structures to a supporting substrate.

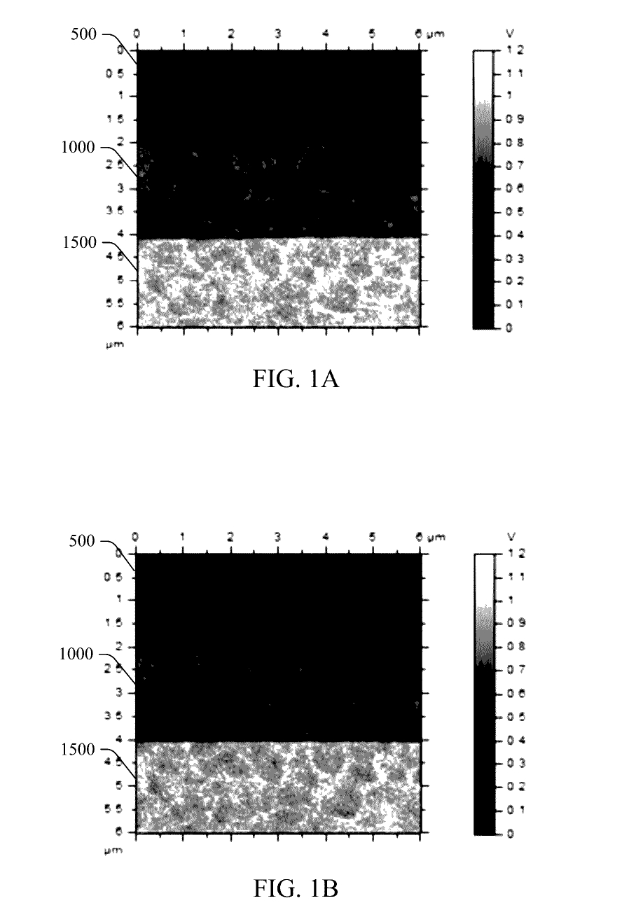

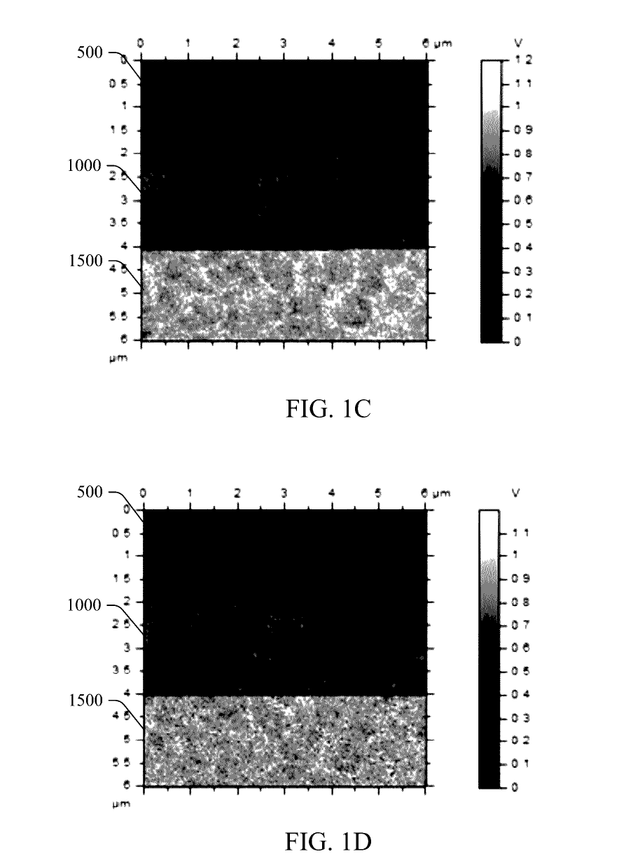

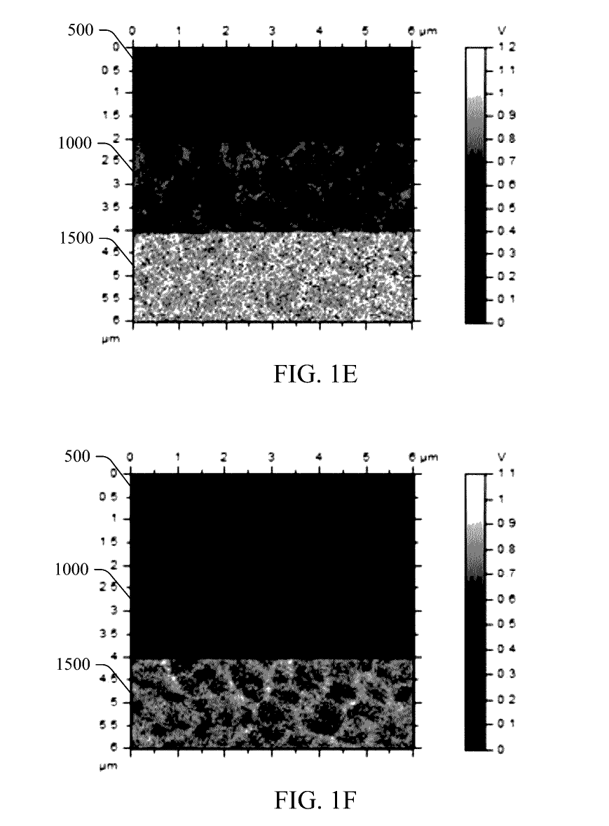

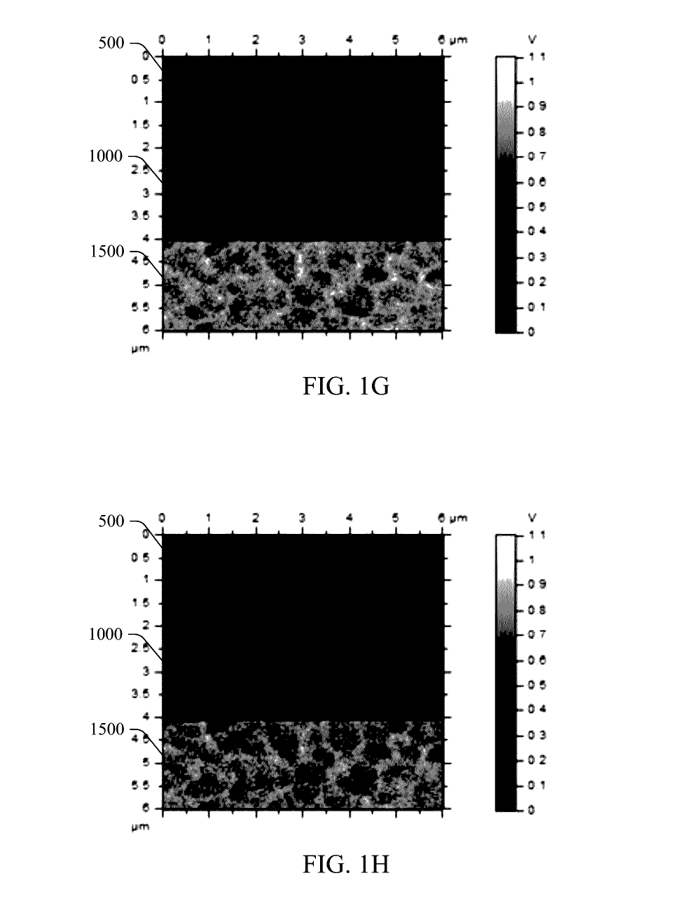

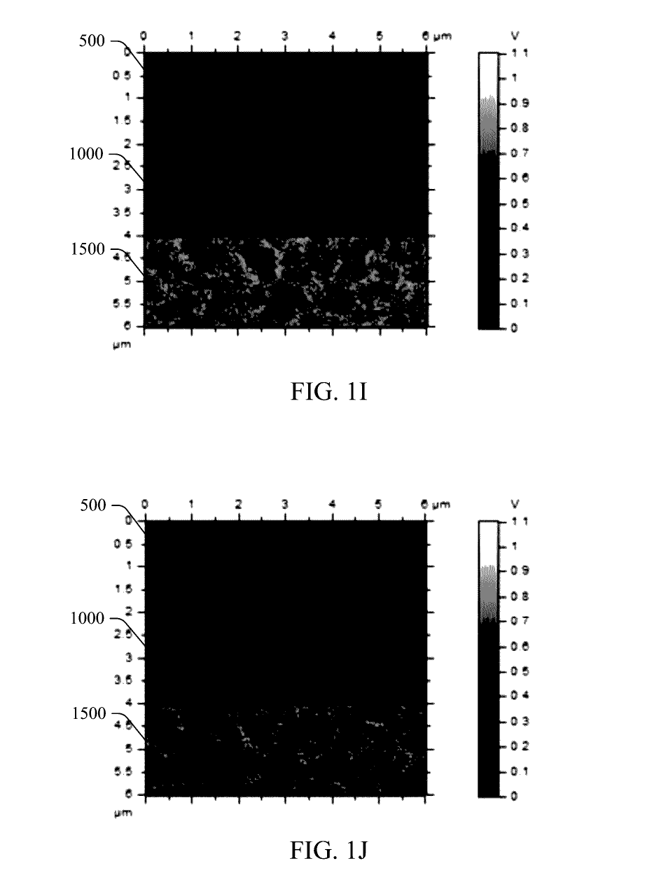

Referring to FIGS. 1A-1J, systematic measurements of piezoelectric responses of a thin PVDF film were performed using piezoelectric force microscope (PFM) under various external DC biases. The results are shown by comparing the imaging contrast under both positive and negative DC biases which were applied between the PFM tip and the backside ground. The diameter of the PFM tip is approximately 30 nm, providing a high spatial resolution. During the measurements, three different forces (500.0 nN, 1000.0 nN, and 1500.0 nN referenced on the figures as 500, 1000, and 1500 respectively) were applied through the PFM tip on the PVDF film. The imaging contrast records the phase difference between the applied AC signal and the piezoelectric effect induced volume change. Higher imaging contrast reflects a higher degree of polarization.

Clearly, an increase of the external force and the DC bias leads to an enhancement of imaging and intensity, which indicates a higher degree of polarization. More interestingly, the PFM images show a clear contrast between the grain and grain boundaries. The imaging contrast evidences the difference of piezoelectricity between the grain and grain boundaries. Under the same external force and DC bias, the grain boundary shows a higher intensity than the grain, indicating a stronger piezoelectric response. The grain boundary experiences a significantly greater strain/stress gradient than the grain. Thus, the significantly enhanced piezoelectric responses from the grain boundary are very likely caused by flexoelectricity.

Comparing the measurements at the positive and negative biases found that the average intensity is lower for measurements with negative biases, indicating the presence of initial polarization of the PVDF film. This is in agreement with the process history of the PVDF film. In addition, the similarity of the imaging contrast between the positive and negative biases means that the imaging contrast is mainly determined by the force applied during the measurements instead of the external biases. Under a 12.0 V bias and 1500.0 nN force, the imaging contrast between the grain and grain boundary is merely distinguished from each other, indicating a saturated polarization occurred. Reversing the bias to negative 12.0 V, 1500 nN is insufficient to saturate the polarization. This is caused by the initial polarization of the PVDF film, whose direction is against the negative bias.

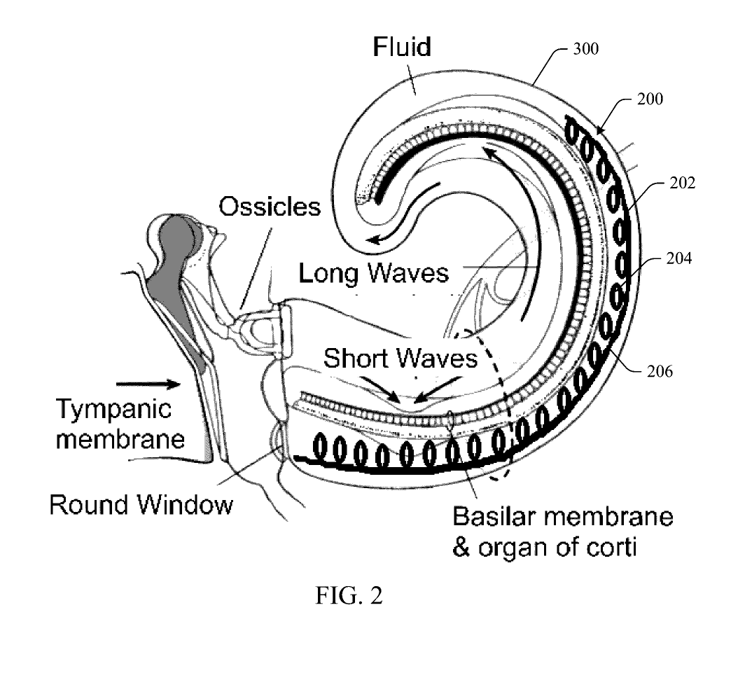

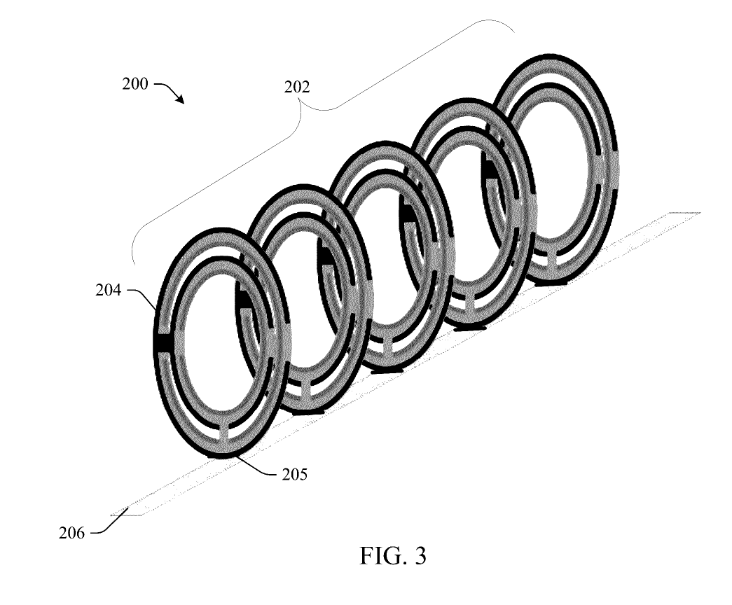

Referring to FIGS. 2 and 3, FIG. 2 is an illustration of one example embodiment of an innovative hearing aid and more specifically, an innovative artificial cochlea device (implant) 200 inserted into a cochlea 300 in accordance with an aspect of the innovation. As mentioned above, the artificial cochlea device 200 works in conjunction with the other normally functioning components of the auditory pathway so that reception of sounds can be much closer to normal hearing.

The artificial cochlea device 200 includes a piezoelectric transducer array 202 that includes ultrathin piezoelectric rings 204, which maximize the flexoelectric effect, attached to a substrate 206, such as but not limited a polymeric substrate. Each ring 204 in the array 202 can be comprised of a single ring or can include multiple (tens to hundreds) of embedded (inner) rings. The rings 204 can include microelectromechanical systems (MEMs) etched cavities in the structure of the ring 204 to further improve non-uniform strain. The substrate 206 can include cavities or recesses 207 that support the piezoelectric rings 204.

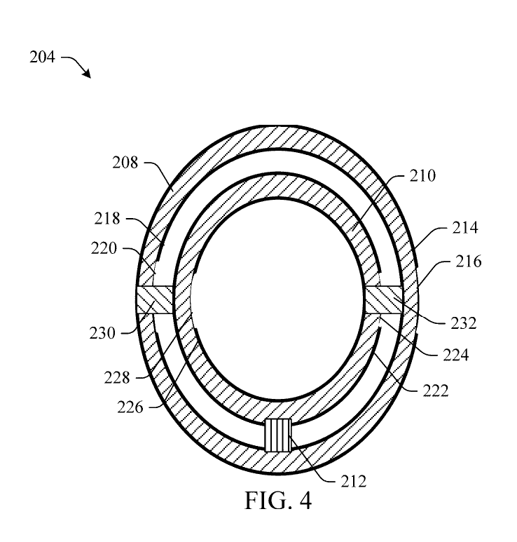

Referring to FIG. 4, each piezoelectric ring 204 includes one or more piezoelectric outer rings 208, one or more piezoelectric inner (embedded) rings 210, and a support 212 that connects the outer and inner rings 208, 210. The outer piezoelectric ring 208 includes a first (first outer) metal layer 214 disposed on an outer surface 216 of the outer ring 208 and a second (first inner) metal layer 218 disposed on an inner surface 220 of the outer ring 208. Similarly, the inner piezoelectric ring 210 includes a third (second outer) metal layer 222 disposed on an outer surface 224 of the inner ring 210 and a fourth (second inner) metal layer 226 disposed on an inner surface 228 of the inner ring 210.

A first electrode (contact) 230 electrically connects the first metal layer 214 of the outer ring 208 to the third metal layer 222 of the inner ring 210 thereby providing a short circuit between the outer surface 216 of the outer ring 210 and the outer surface 224 of the inner ring 210. A second electrode (contact) 232 electrically connects the second metal layer 218 of the outer ring 208 to the fourth metal layer 226 of the inner ring 210 thereby providing a short circuit between the inner surface 220 of the outer ring 208 and the inner surface 228 of the inner ring 210.

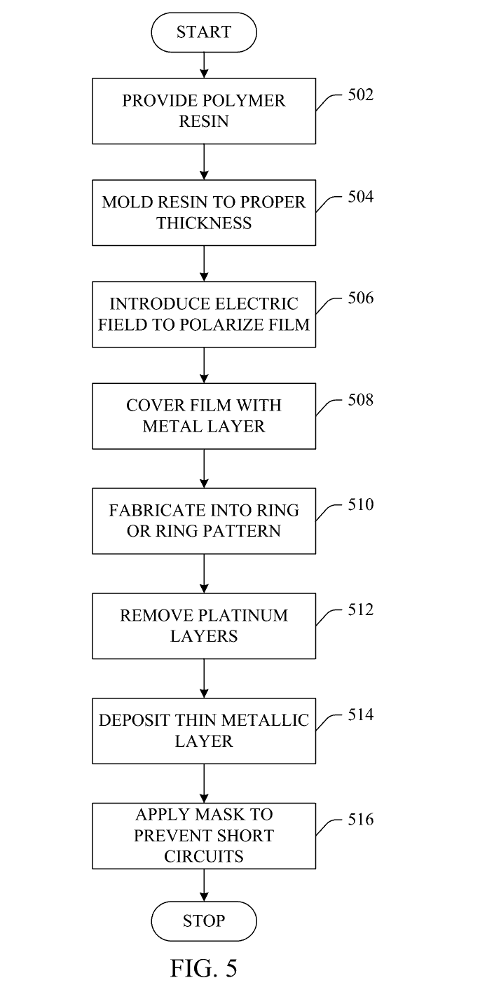

Referring to FIG. 5, the subject innovation can be fabricated using MEMs technology, starting at 502 with a polymer base formed from a polymer resin. At 504, the resin can be molded to the proper thickness and size for further processing, where the thickness can be reduced to approximately 100 nm to vastly improve the output of the flexoelectric effect. At 506, a strong electric field is introduced that serves to polarize the film, creating electric dipoles that give the material a permanent piezoelectric property thus, forming a piezoelectric film. At 508, the thin piezoelectric film is covered with a thin biocompatible metal layer on both sides can be attached on a silicon wafer support substrate that is coated with a thin photoresist layer to adhere the piezoelectric film. At 510, the piezoelectric film can then be fabricated into a single ring or embedded ring pattern through photolithography and reactive ion etching, using the metal as a hard mask. At 512, the remaining platinum layers on both sides can be removed by using wet chemical etching. At 514, a thin metallic layer can be deposited using sputtering coater for uniform coverage and to connect the multiple electrical signals of the multiple rings. At 516, to prevent short circuits, a second mask can be designed and applied to an upper surface to protect the upper surface and the electrical connections.

A similar procedure can be repeated to connect of all the bottom surfaces using a third mask. Tens to hundreds of these ring structures can be created using the above procedure. In one or more embodiments of the device, the multitude of transducer rings can be mechanically isolated from each other and attached to a biocompatible substrate, being spread along the holding structure to stimulate different loci of nerves along the cochlear. The spacing between these rings can be approximately 27 um to reach equivalent frequency selectivity of a working human cochlea.

Referring back to FIG. 2, the size of the innovative artificial cochlea device 200 is such that it can be non-traumatically inserted into the cochlea. More specifically, a thickness of each ring is less than 1 micron, a diameter of each of the rings 204 is approximately 0.6 mm, a total diameter of the array 200 is less than 1 mm, a length of the substrate 206 ranges from 10-30 mm, and, as mentioned above, the space between each of the plurality of piezoelectric rings 204 mounted on the substrate 206 is approximately 27 um. The device is to be inserted near the round window of the cochlea. The oval window is avoided so as to not damage the ossicles. The implementation of the innovation requires that the outer and middle ear be functional, thus the stapes connected to the oval window needs preservation. Surgical techniques can seal the round window region after implantation and maintain the natural fluid levels within the cochlea.

In many cases of sensorineural deafness, the low frequency registers of the inner ear still function while the high and middle frequency registers no longer properly respond. In such instances, the innovative artificial cochlea device can preserve the functionality of these low frequency components while restoring lost high frequency hearing. The length of the device can be altered to a longer or short length based on the needs of the individual. By changing the length, the device can affect auditory nerves specific to certain frequency ranges. For example, if an individual suffers hearing loss above 2000 Hz, a device can be fabricated with rings near to loci of nerves with frequencies above 2000 Hz and inserted only the depth corresponding to these frequencies.

Embodiments of the artificial cochlea can be implanted in a selected portion of the cochlear turn and can function by stimulating a smaller region of the nerves in the inner ear. The preservation of the outer and middle ear results in a less intrusive measure than the currently used series of electrodes. The device in accordance with the subject innovation can be completely contained within the cochlea, whereas conventional devices necessitate connectors between the electrode array and receiver structure. The design of embodiments of the subject innovation can readily accommodate future improvements in surgical technology to achieve improved hearing resolution due to the transducer sizes and quantity.

Aspects of the subject innovation can employ or comprise thin film, biocompatible piezoelectric ring-shaped devices. In various embodiments, devices as described herein can be inserted into the cochlea in an atraumatic manner and can rest along the under surface of the proximal portion of the basilar membrane. In various aspects, flexoelectric-enhanced rings with vast electrical output stimulate the neurons of the auditory nerve with frequency selectivity comparable to a functional human cochlea. The subject innovation can act as a replacement for failing basilar membrane or hair cells of the cochlea, working with the outer and middle ear mechanism, which can provide additional amplification.

Compared to conventional hearing aids and cochlear implants, an artificial cochlea according to aspects of the subject innovation can provide multiple advantages. Hearing aids and cochlear implants require external microphones and an outside power source, which is not required by the subject innovation and which is totally implantable. As opposed to relying on software to generate electric signals and determine frequency as in conventional artificial cochleas, this can be done simultaneously by the ring structures. Additionally, hearing aids and cochlear implants can cause possible physical discomfort from the device and raise cosmetic concerns, both of which can be avoided with the subject innovation. The possibilities of difficulty hearing in noisy environments or distortion of sounds which occur with hearing aids and cochlear implants can be reduced with an artificial cochlea as described herein, and the likelihood of a restoration of normal hearing is increased. Additionally, in contrast with cochlear implants, the subject innovation does not destroy any residual hearing remaining prior to implantation and can be a minimally invasive procedure. Among other groups, the subject innovation can be of substantial benefit to patients with moderate to severe nerve deafness caused by age or noise exposure, which is the overwhelming majority of the hearing impaired worldwide.

What has been described above includes examples of the innovation. It is, of course, not possible to describe every conceivable combination of components or methodologies for purposes of describing the subject innovation, but one of ordinary skill in the art may recognize that many further combinations and permutations of the innovation are possible. Accordingly, the innovation is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A hearing device comprising:
a plurality of piezoelectric rings; and
a polymeric substrate,
wherein the plurality of piezoelectric rings are attached to the polymeric substrate.

2. The hearing device of claim 1, wherein each of the plurality of piezoelectric rings includes at least one outer ring, at least one inner ring, and a support that connects the at least one outer ring to the at least one inner ring.

3. The hearing device of claim 2 further comprising a first metal layer disposed on an outer surface of the outer ring, a second metal layer disposed on an inner surface of the outer ring, a third metal layer disposed on an outer surface of the inner ring, and a fourth metal layer disposed on an inner surface of the inner ring.

4. The hearing device of claim 3 further comprising a plurality of first electrodes connected to the first metal layer of the outer ring and the third metal layer of the inner ring.

5. The hearing device of claim 4 further comprising a plurality of second electrodes connected to the second metal layer of the outer ring and the fourth metal layer of the inner ring.

6. The hearing device of claim 1, wherein a thickness of each of the plurality of piezoelectric rings is less than 1 micron.

7. The hearing device of claim 1, wherein a space between each of the plurality of piezoelectric rings mounted on the substrate is approximately 27 um.

8. The hearing device of claim 1, wherein a diameter of each of the plurality of piezoelectric rings ranges from 0.4 mm to 0.8 mm and a total diameter of the piezoelectric transducer array is less than 1 millimeter.

9. The hearing device of claim 1, wherein a length of the polymeric substrate ranges from 10 to 30 millimeters.

10. An artificial cochlea comprising:
a piezoelectric transducer array including:
a plurality of piezoelectric rings; and
a polymeric substrate,
wherein the plurality of piezoelectric rings are attached to the polymeric substrate.

11. The hearing device of claim 10, wherein each of the plurality of piezoelectric rings includes a plurality of outer rings and a plurality of embedded rings, and a plurality of supports that connect the plurality of outer rings to the plurality of embedded rings.

12. The hearing device of claim 11 further comprising a first metal layer disposed on an outer surface of each of the plurality of outer rings, a second metal layer disposed on an inner surface of each of the plurality of outer rings, a third metal layer disposed on an outer surface of each of the plurality of inner rings, and a fourth metal layer disposed on an inner surface of each of the plurality of inner rings.

13. The hearing device of claim 12 further comprising a plurality of first electrodes connected to the first metal layer of the plurality of outer rings and the third metal layer of the plurality of inner rings.

14. The hearing device of claim 13 further comprising a plurality of second electrodes connected to the second metal layer of the plurality of outer rings and the fourth metal layer of the plurality of inner rings.

15. The hearing device of claim 10, wherein a diameter of each of the plurality of piezoelectric rings ranges from 0.4 mm to 0.8 mm and a total diameter of the piezoelectric transducer array is less than 1 millimeter.

16. A method comprising:
providing a polymer resin;
molding the resin into a predetermined thickness;
introducing an electric field to the resin;
forming a piezoelectric film;
covering the piezoelectric film with a metal layer; and
forming the piezoelectric film into a plurality of piezoelectric rings using the metal layer as a first mask.

17. The method of claim 16, wherein forming the piezoelectric film into a plurality of piezoelectric rings using the metal layer as a first mask includes etching the plurality of piezoelectric rings in the piezoelectric film.

18. The method of claim 17 further comprising etching a plurality of embedded piezoelectric rings into each of the plurality of piezoelectric rings.

19. The method of claim 18 further comprising depositing a metallic layer on each of the plurality of piezoelectric rings and each of the embedded plurality of piezoelectric rings to provide an electrical connection between the each of the plurality of piezoelectric rings and each of the embedded plurality of piezoelectric rings.

20. The method of claim 19 further comprising applying a second mask to each of the plurality of piezoelectric rings and each of the embedded plurality of piezoelectric rings to protect the electrical connection.
